(19) United States
(12) Patent Application Publication
(10) Pub. No.: US 2021/0022621 A1
(43) Pub. Date: Jan. 28, 2021

(54) HEART FAILURE DECOMPENSATION MONITORING

(71) Applicant: West Affum Holdings Corp., Grand Cayman (KY)

(72) Inventors: Joseph L. Sullivan, Kirkland, WA (US); Pamela F. Breske, Newcastle, WA (US); Phillip D. Foshee, JR., Woodinville, WA (US); Laura M. Gustavson, Redmond, WA (US); Regina New, Richmond, VA (US); Krystyna Szul, Seattle, WA (US)

(73) Assignee: West Affum Holdings Corp., Grand Cayman (KY)

(21) Appl. No.: 16/940,259

(22) Filed: Jul. 27, 2020

Related U.S. Application Data
(60) Provisional application No. 62/878,975, filed on Jul. 26, 2019.

(51) Int. Cl. A61B 5/0205; A61B 5/00; A61B 5/11; A61B 5/113

(52) U.S. Cl. CPC A61B 5/02055; A61B 5/6891; A61B 5/0004; A61B 2560/0214; A61B 5/747; A61B 5/1116; A61B 5/1135; A61B 5/7475

(57) ABSTRACT

A sensor substrate to detect heart failure decompensation comprises a substrate structure, wherein the substrate structure is configured to have a patient sit, recline, or lie on a surface of the substrate structure, one or more sensors in the substrate structure, wherein the one or more sensors are configured to detect a plurality of parameters of the patient when the patient is in contact with the substrate structure, an interface in the substrate structure to receive an output from the one or more sensors, and a processor, wherein the processor is configured to detect heart failure decompensation in the patient from the plurality of parameters detected by the one or more sensors when the patient is in contact with the substrate structure.

### CROSS-REFERENCE TO RELATED APPLICATIONS

The present application claims the benefit of U.S. Provisional Application No. 62/878,975 (C00003626.USP1) filed Jul. 26, 2019. Said Application No. 62/878,975 is hereby incorporated herein by reference in its entirety.

### BACKGROUND

Heart failure decompensation can be described as a sudden worsening of the symptoms of heart failure, characterized by difficulty breathing, which can be while performing a low level activity like walking, or while lying down, waking up from sleeping gasping for air, fluid build-up or swelling of limbs due to acute pulmonary edema. Late signs of heart failure can include tachycardia, pedal edema, increased jugular venous pressure, respiratory crackles, S3 gallop or a third heart sound, and/or peripheral capillary oxygen saturation (SpO2) levels. For example, see Inamdar, A. R. et al. “Heart Failure: Diagnosis, Management and Utilization”, J. Clin. Med. (2016, 5, 62) https://www.ncbi.nlm.nih.gov/pmc/articles/PMC4961993/. Acute decompensated heart failure (ADHF) is a serious condition and a leading cause of hospital admissions of patients over the age 65, and patients with ADHF require urgent medical assessment and treatment. For example, see Ali D. et al. “Inpatient Monitoring of Decompensated Heart Failure: What Is Needed?”, Curr. Heart Fail Rep. (Aug. 12, 2017) 14:393-397 https://www.ncbi.nlm.nih.gov/pmc/articles/PMC5597700/.

### DESCRIPTION OF THE DRAWING FIGURES

Claimed subject matter is particularly pointed out and distinctly claimed in the concluding portion of the specification. However, such subject matter may be understood by reference to the following detailed description when read with the accompanying drawings in which:

FIG. 1 is a diagram of a heart failure decompensation monitoring system including a sensor substrate that can be used to monitor and detect heart failure decompensation in accordance with one or more embodiments.

FIG. 2 is a diagram of a control module that can be used in cooperation with the sensor substrate of FIG. 1 in accordance with one or more embodiments.

FIG. 3 is a diagram of additional sensors that are not part of the sensor substrate in accordance with one or more embodiments.

FIG. 4 is a diagram of a heart failure decompensation monitoring system capable of communicating with a remote health care provider in accordance with one or more embodiments.

FIG. 5 is a diagram of a method to detect heart failure decompensation using sensor substrate of a monitoring system in accordance with one or more embodiments.

It will be appreciated that for simplicity and/or clarity of illustration, elements illustrated in the figures have not necessarily been drawn to scale. For example, the dimensions of some of the elements may be exaggerated relative to other elements for clarity. Further, if considered appropriate, reference numerals have been repeated among the figures to indicate corresponding and/or analogous elements.

### DETAILED DESCRIPTION

In the following detailed description, numerous specific details are set forth to provide a thorough understanding of claimed subject matter. It will, however, be understood by those skilled in the art that claimed subject matter may be practiced without these specific details. In other instances, well-known methods, procedures, components and/or circuits have not been described in detail.

In the following description and/or claims, the terms coupled and/or connected, along with their derivatives, may be used. In particular embodiments, connected may be used to indicate that two or more elements are in direct physical and/or electrical contact with each other. Coupled may mean that two or more elements are in direct physical and/or electrical contact. However, coupled may also mean that two or more elements may not be in direct contact with each other, but yet may still cooperate and/or interact with each other. For example, “coupled” may mean that two or more elements do not contact each other but are indirectly joined together via another element or intermediate elements. Finally, the terms “on,” “overlying,” and “over” may be used in the following description and claims. “On,” “overlying,” and “over” may be used to indicate that two or more elements are in direct physical contact with each other. It should be noted, however, that “over” may also mean that two or more elements are not in direct contact with each other. For example, “over” may mean that one element is above another element but not contact each other and may have another element or elements in between the two elements. Furthermore, the term “and/or” may mean “and”, it may mean “or”, it may mean “exclusive-or”, it may mean “one”, it may mean “some, but not all”, it may mean “neither”, and/or it may mean “both”, although the scope of claimed subject matter is not limited in this respect. In the following description and/or claims, the terms “comprise” and “include,” along with their derivatives, may be used and are intended as synonyms for each other.

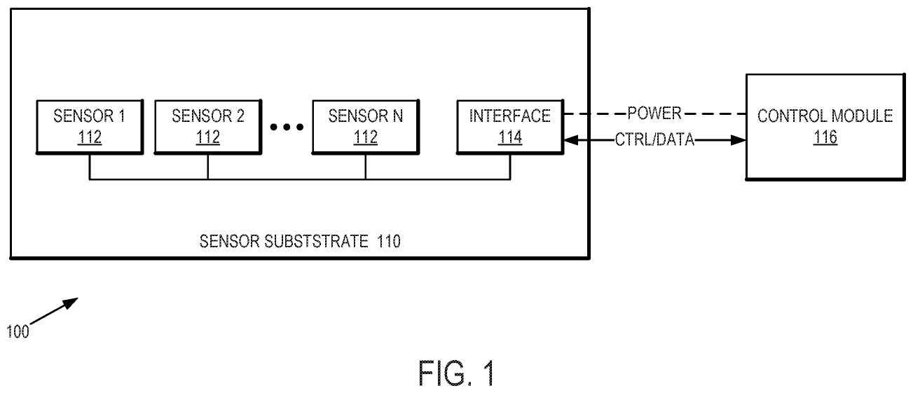

Referring now to FIG. 1, a diagram of a heart failure decompensation monitoring system including a sensor substrate that can be used to monitor and detect heart failure decompensation in accordance with one or more embodiments will be discussed. FIG. 1 shows a monitoring system 100 that is capable of monitoring for and detecting heart failure decompensation so that the needed medical assessment or treatment can be started. Monitoring system 100 includes a sensor substrate 110 having one or more sensors 112, up to N number of sensors to monitor and detect various clinical parameters and symptoms of heart failure in a patient, for example wherein the patient is in a home environment. Monitoring system 100 can transmit the detected parameters and any related symptoms to the patient's health care provider. In some embodiments, the parameters monitored by the sensors 112 of sensor substrate and transmitted to the health care provider can include weight, blood pressure, heart rate, or oxygen saturation. In addition, information regarding medications, symptoms, or responses to prompts issued to the patient by the system can be transmitted to the health care provider. The health care provider can use this information to apply or titrate therapy provided to the patient. In some embodiments, utilization of monitoring system 1100 of FIG. 1 can reduce costs, reduce patient mortality or hospitalizations, or can be equivalent to a telephone calls or in home visits by a nurse or other health care provider.

In one or more embodiments, monitoring system 100 can be configured or adapted to operate while the patient is reclining. For example, the patient may utilize monitoring system 100 while lying in a bed or in a reclining chair at home, at a doctor's office, hospital, assisted-living facility, and so on. In some embodiments, the sensor substrate 110 can include multiple sensors 112 and can be provided in various form factors for example a pad, a mattress, a cushion, a pillow, a liner, a cover, and so on. Sensor substrate 110 can be placed on a bed or a chair, or alternatively sensor substrate 110 can be incorporated into a bed or a chair such as comprising a mattress or a cushion or a portion of a mattress or a cushion. In some embodiments, sensor substrate 110 can be ergonomically designed for the patient's comfort. In the case of non-mattress embodiments, sensor substrate 110 can be designed or configured for ease of installation on a bed or chair so that sensor substrate 110 can remain in a desired position even while the patient is moving or shifting in the bed or chair. For example, sensor substrate can include straps, fasteners, hook and loop fasteners, and so on, to maintain sensor substrate 110 in a fixed position or location on the bed or chair. When used with a bed or incorporated into a bed, sensors 112 of sensor substrate 110 can monitor various parameters of the patient, such as heart rate or respiration rate, while the patient is sleeping, although scope of the disclosed subject matter is not limited in this respect.

In some embodiments, sensor substrate 110 can include one or more pressure or strain sensors in various locations in sensor substrate 110 to monitor and determine the patient's posture and movement, expansion and contraction of the patient's chest during breathing, patient weight, or weight distribution attributable to various parts of the patient's limbs such as the patient's lower legs. For example, if a patient's lower legs have fluid build-up due to edema, such sensors located near the lower portion of sensor substrate 110 on which the patient's legs are placed can sense an increase in weight or surface area due to swelling of the legs in contact with the lower portion of sensor substrate 110. In some embodiments, one or more sensors 112 can be used to detect and monitor paradoxical rhythm from movement of the patient's chest compared with movement of the patient's abdomen. In some embodiments, the pressure or strain sensors may be used to determine the patient's heart rate, respiration rate and/or other vital signs as disclosed in U.S. Pat. No. 8,444,558. In some embodiments, one or more sensors 112 can include one or more accelerometers. The accelerometer(s) may be used to determine either alone or with information from other sensor(s) the patient's posture, sleep incline or angle, the patient's angle of sitting, reclining, and/or patient movement or activity. In some embodiments, the patient's sleep incline or angle may be received or determined from data received from an adjustable bed having communication functionality.

In some embodiments, the sensors 112 of sensor substrate 110 can include one or more temperature sensors to measure the temperature of various parts of the patient's body. In some embodiments ambient temperature sensors can also be included in sensor substrate 110. In such embodiments, the temperature sensors can monitor and detect temperature variations across the patient's body to facilitate comparison to variations in the patient's body temperature common with heart failure. Multiple ambient or environmental temperature sensors along with their positions in sensor substrate 110 can be used to track potential effects of the environmental temperature on the patient temperature at various locations of the patient's body. In some embodiments, one or more of the temperature sensors can comprise infrared sensors, although the scope if the disclosed subject matter is not limited in this respect.

In some embodiments, one or more of the sensors 112 of sensor substrate 110 can include one or more microphones. The microphone sensors can be used, for example, to detect respiratory crackles or other respiratory system sounds (e.g., dyspnea, coughing, wheezing, rales, or, when combined with sleep angle information orthopnea), heart sounds, voice commands from the patient, and do on. In one or more examples, detection of respiratory crackles can be performed according to Reyes, B. A. et al. “A Smartphone-Based System for Automated Bedside Detection of Crackle Sounds in Diffuse Interstitial Pneumonia Patients”, Sensors (2018) 18, 3813 https://www.ncbi.nlm.nih.gov/pmc/articles/PMC6263477 which discloses a system using a smart phone and adhesive sensors to detect crackles, or according to Grønnesby, M. et al. “Machine Learning Based Crackle Detection in Lung Sounds”, (May 31, 2017) https://www.researchgate.net/publication/317299980_Machine_Learning_Based_Crackle_Detection_in_Lung_Sounds which discloses using machine-learning to classify crackle from stethoscope recordings.

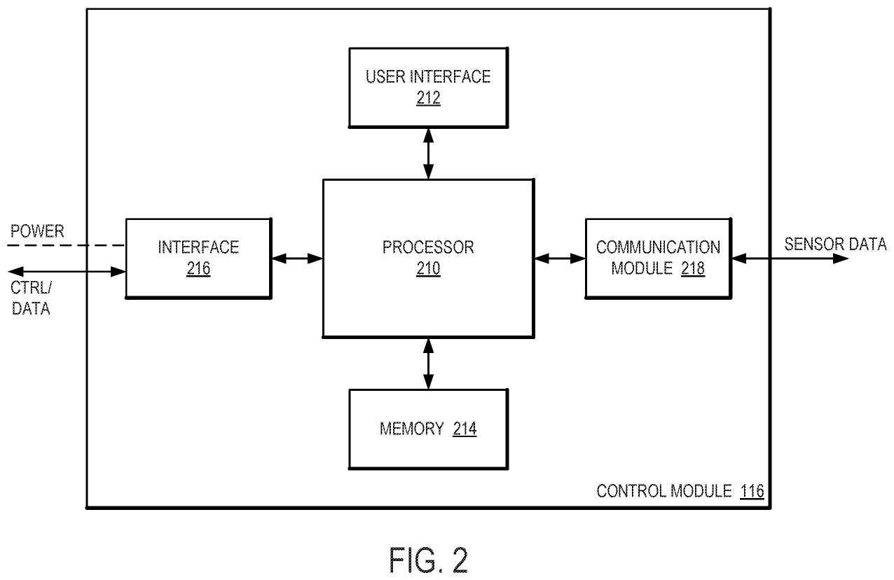

In one or more embodiments, sensor substrate 110 can include an interface 114 to communicate with a control module 116. Control module 116 can be provided in a housing that is external to sensor substrate 110 and can be used to send or received control signals or data to or from sensor substrate 110. In addition, control module 116 optionally can be configured to provide power to sensor substrate 110 including powering one or more of sensors 112 or powering interface 114. In some embodiments, the control functions of control module 116 can be included within or embedded withing sensor substrate 110. In other embodiments, control module 116 can be partially or wholly external to sensor substrate 110, and can communicate with interface 114 of sensor substrate 110 using a wired or a wireless link. In some embodiments, control module 116 can be embodied as or integrated in a smart phone, tablet, smart watch, smart speaker, smart hub, or personal computer, or the like. For example, interface 114 can comprise a Bluetooth or Zigbee interface and associated circuitry to communicate with an external device used to control sensor substrate 110 and provide the functions of control module 116. In another example, interface 114 can comprise a Universal Serial Bus (USB) interface or the like to communicate with an external control module 116. Details of an example control module 116 are shown in and described with respect to FIG. 2, below.

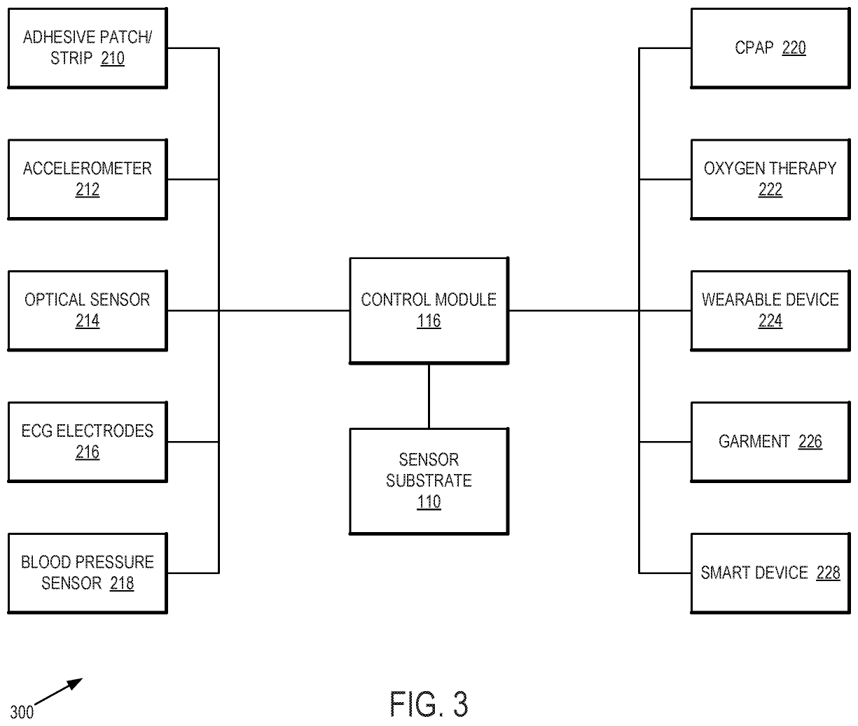

Referring now to FIG. 2, a diagram of a control module that can be used in cooperation with the sensor substrate of FIG. 1 in accordance with one or more embodiments will be discussed. As discussed with respect to FIG. 1 above, control module 116 can be partially or wholly within sensor substrate 110, or can be partially or wholly external to sensor substrate 110, for example where control module 116 includes its own housing or other external device. Control module 116 can include a processor 210, a user interface 212, a memory 214, an interface 216 for sensor substrate 110, or a communication module 218. User interface 212 can include one or more keys or buttons, a display, a touch screen display, one or more speakers, one or more microphones, and so on, to allow a patient or user to operate and control sensor substrate 110. Memory 214 can be used to store algorithms for operating sensor substrate 110, communication protocols, sensor data obtained from one or more of sensors 112, processing results, reports, and so on. Interface 216 can be used to communicate with sensor substrate 110 and to obtain data from one or more of sensors 112. Optionally, power can be provided to sensor substrate 110 via interface 216, for example where interface comprises a USB compliant interface or the like capable of providing power to an external device over a USB connection. Communication module 218 can include a network interface such as Ethernet, a wireless local area network (WLAN) circuit compliant with an institute of Electrical and Electronics Engineers (IEEE) 802.11, or a wireless wide area network (WWAN) circuit compliant with a Third Generation Partnership Project (3GPP) standard. Communication module 218 can transmit data or reports obtained by one or more sensors 112 of sensor substrate 110 to a remote location such as a doctor's office via a network such as the Internet for viewing and analysis by an appropriate health care provider. In some embodiments, communication module 116 can receive patient data from one or more additional sensors that are external to sensor substrate 110, for example as shown in and described with respect to FIG. 3, below.

Referring now to FIG. 3, a diagram of additional sensors that are not part of the sensor substrate in accordance with one or more embodiments will be discussed. As shown in FIG. 3, in some embodiments monitoring system 100 of FIG. 1 can include one or more additional sensors that are not part of sensor substrate 110.

For example, some additional sensors can be provided in an adhesive patch or strip 210 applied to the patient. The additional sensors can include more accelerometers 212, optical sensors 214 such as pulse oximeter (SpO2) sensors, electrocardiogram (ECG) electrodes 216, blood pressure measurement devices or sensors 218, Doppler or ultra-wide band (UWB) sensors for detecting patient motion, breathing, heart rate (e.g., as disclosed in “Detection of Breathing and Heart Rates in UWB Radar Sensor Data using FVPIEF Based Two-Layer EEMD” IEEE Sensors Journal PP(99):1-1 Oct. 2018), and so on. In such embodiments, one or more sensors 112 of sensor substrate 110 can comprise a transmitter, receiver, or transceiver to transmit doppler or UWB pulses or signals into the thorax of the patient, to the receive the reflected or echo pulses or signals back from the patient's thorax to detect patient motion, heart rate, breathing, or apnea. In some examples, processor 210 can comprise a digital signal processor (DSP), or an additional DSP device can be provided in sensor substrate 110 or control module 116, to facilitate processing and analysis of doppler or UWB signals, for example to perform fast Fourier transform (FFT) algorithms, and the scope of the disclosed subject matter is not limited in this respect.

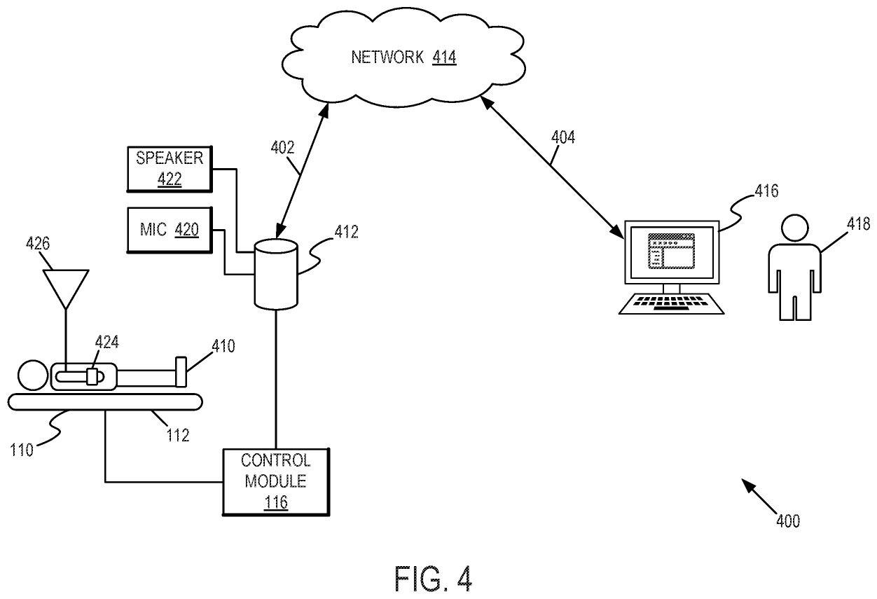

In one or more embodiments, additional sensors can be disposed in or on a home or personal medical device. A home or personal medical device can comprise, for example, a breathing assist device such as a continuous positive airway pressure (CPAP) machine 220 or oxygen therapy device 222, or in a wearable device 224 or garment 226 such as a watch, a ring, an armband, a leg band, a band worn about the torso or abdomen, a sock, a shirt, a vest, a fitness tracker, blood pressure cuff, and so on. In some embodiments, an additional sensor can comprise or be part of an external device or smart device 228 with sensors, for example a smartphone located near the patient, a smart speaker such as AMAZON ALEXA or ECHO device or a GOOGLE HOME HUB device, a charging station for the components of the monitoring system, and so on. An example of how sensor substrate 110 optionally with one or more additional sensors or devices can be used to provide a heart failure decompensation monitoring system is shown in and described with respect to FIG. 4 below.

Referring now to FIG. 4, a diagram of a heart failure decompensation monitoring system capable of communicating with a remote health care provider in accordance with one or more embodiments will be discussed. Heart failure decompensation monitoring system 400 can include sensor substrate 110 having one or more internal sensors 112 and can couple with control module 116 as described herein. Control module 116 can link to a smart device 412 such as a smart speaker or home hub, smart phone, tablet, personal computer, home health care device, and so on. In some examples, smart device 412 can comprise smart device 228 of FIG. 3. In some examples, smart device 412 can include one or more additional sensors external to sensor substrate. In some examples, smart device 412 can include a transmitter and a receiver, or a transceiver, to transmit and receive signals to and from patient 410 for Doppler signal analysis and processing. Smart device 412 can include one or more microphones 420 and one or more speakers 422 to allow patient 410 interact and control smart device 412. Furthermore, smart device 412 can connect with a remote health care system 416 located remotely from patient 410, for example in another room, in another building, in another city, or in another state or country from the location patient 410. In some examples, smart device 412 can connect to network 414 via link 402. Network 414 can comprise a local area network or a wide area network, and can comprise any combination of wired links or wireless links. In some examples, network 414 can comprise the Internet. Similarly, remote health care system 416 can connect to network via link 414 which can comprise a link or connection similar to link 402. Using such an arrangement, a remote health care provider or professional 418 can monitor and review the data obtained from the sensors 112 of sensor substrate 110.

In some examples, health care provider 418 can control the operation of sensor substrate 110 remotely from health care system 416. In some examples, health care system 416 can comprise a network, a server, a computer workstation, or a smart device such as smart device 228 of FIG. 3 or smart device 412 of FIG. 4. The arrangement of system 400 can allow health care provider 418 to communicate with patient 410. For example, health care provider 418 can ask a question to patient 410 by speaking into health care system 416, and the voice of health care provider 418 can be transmitted via network 414 to be heard by patient 410 via speaker 422. The verbal response of patient 410 can then be received with microphone 420 and transmitted to health care provider 418 via network 414. In such embodiments, health care system 416 can include a microphone and speaker, or a headset including a microphone and speaker can be worn by health care provider 418. Using such an arrangement, health care provider 418 can apply or titrate an appropriate medicine or other treatment 426 to patient 410. The medicine or treatment 426 can be titrated or applied by the patient himself, by another person or healthcare worker in the vicinity of patient 410, or by a device coupled with control module 116 or smart device 412 that is configured to automatically administer medicine or treatment 426 via a command provided by health care provider 418 via network 414 using health care system 416. By using sensor substrate 110 as part of heart failure decompensation monitoring system 400, heart failure decompensation in patient 410 can be detected more easily and more accurately when the patient 410 is remotely located with respect to health care provider 418. Furthermore, heart failure decompensation can be detected in patient 410 by monitoring system 400 when patient 410 is at home allowing detection and treatment of heart failure decompensation to occur earlier than it would otherwise be detect and treated to reduce hospitalization or mortality. In some embodiments, sensor substrate 110 can comprise a portable structure such as a pad or a cushion so that the patient 410 can use sensor substrate in a variety of locations and usage scenarios. For example, sensor substrate 110 can be placed on top of a bed or mattress so that patient 410 can lie on sensor substrate 110 when the patient 410 is sleeping. Then when the patient 410 is awake, sensor substrate 110 can be placed on a chair or recliner so that the patient 410 sit on sensor substrate 110 when the patient 410 is sitting in the chair or reclining in the recliner. Such a portable sensor substrate 110 can be used in other scenarios such as placed on a floor, a therapy table or cart, and so on, and in general the body of the patient 410 can be in contact with the portable sensor substrate 110 during use. In some embodiments, the sensor substrate 110 can be shaped or include structures (e.g., ridges, indentations, straps, cuffs, etc.) that facilitate positioning, aligning, receiving, stabilizing, etc. the patient's body (or portions thereof) so that the certain sensors are positioned to output “sensed” signals with sufficient quality or accuracy. For example, the structure of sensor substrate 110 can comprise a first upper body portion that generally matches the size and shape of a patient's thorax, and can have a second lower body portion that generally matches the size and shape of a patient's legs. Thus, when the patient lies, reclines, or sits on the substrate structure, the patient can place his or her upper body on the thorax portion, and place his or her legs each of the leg portions. The upper body portion of the substrate structure can include heart rate and breathing sensors to detection patient heart rate and respiration rate, and the lower body portion can have weight or strain sensors to detect edema in the patient's legs. In other embodiments, sensor substrate 110 can be embedded or integrated in another structure, for example embedded in a mattress, or a seat cushion or a seat back of a chair, recliner, couch, and so on. It should be noted that these are merely example structures for housing sensor substrate 110, and the scope of the disclosed subject matter is not limited in these respects.

In some embodiments, heart failure decompensation monitoring system 400 can include one or more additional sensors or devices that are external to sensor substrate 110. For example, patient 410 can utilize an external device 424 that can include one or more sensors or one or more external devices as shown in and described with respect to FIG. 3 above. External device can include additional sensors to provide data in addition to the data monitored by the internal sensors 112 of sensor substrate 110. This additional data can be transmitted to control module 116 or to smart device 412 and can be transmitted to health care system 416 via network 414, either separately from the data obtained by the sensors 112 of sensor substrate 110, or combined with the data obtained by the sensors 112 of sensor substrate 110, for example in a combined readout or report.

It should be noted that although FIG. 4 shows a heart failure decompensation monitoring system 400 that utilizes a smart device 412, in some embodiments smart device 412 need not be used or provided. For example, control module 116 can itself include the circuitry and devices to function as smart device 412 and can include one or more microphones 420 or one or more speakers 422 to allow patient 410 to interact and communicate with remote health care provider 418 via network 414. In some further examples, sensor substrate 110 can itself include the circuitry and devices to function as smart device 412 and can include one or more microphones 420 or one or more speakers 422 to allow patient 410 to control and interact with control module 116, or to interact and communicate with remote health care provider 418 via network 414. In some examples, control module 116 or sensor substrate 110 includes the circuitry and devices to interact with or control one or more additional external sensors or devices of device 424. In yet further examples, control module 116 or sensor substrate 110 can communicate with device 424 which can itself include the circuitry and devices to function as smart device 412 and can include one or more microphones 420 or one or more speakers 422 to allow patient 410 to interact and communicate with remote health care provider 418 via network 414.

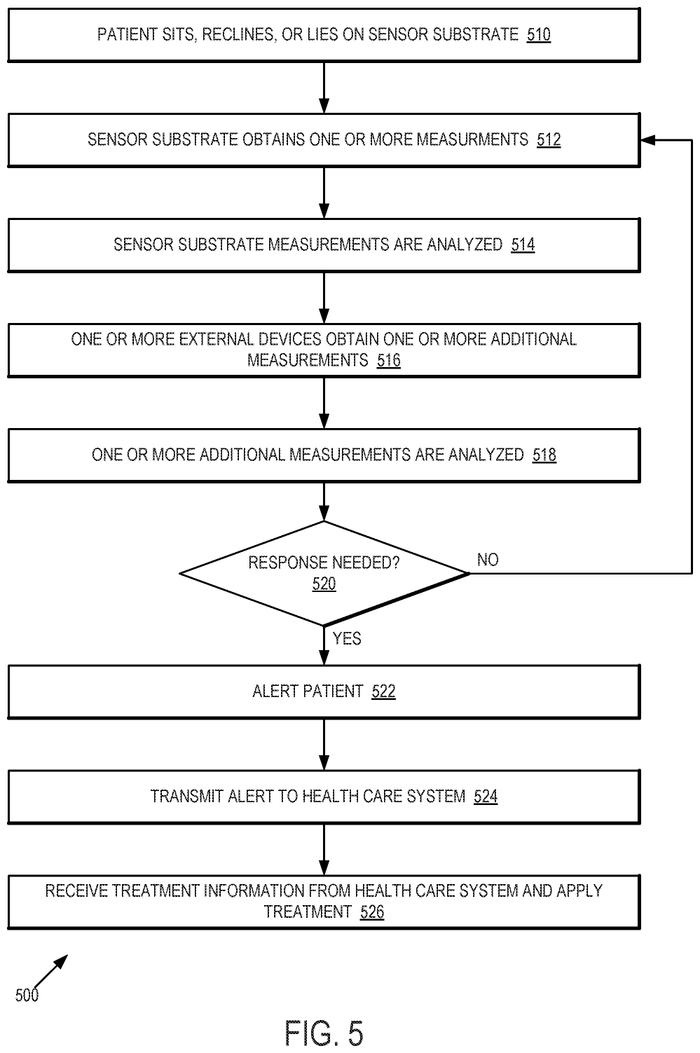

Referring now to FIG. 5, a diagram of a method to detect heart failure decompensation using a sensor substrate of a monitoring system in accordance with one or more embodiments will be discussed. It should be noted that although FIG. 5 shows one particular order for the operations of method 500, the operations of method can be in various other orders, and can include more or fewer operations than shown, and the scope of disclosed subject matter is not limited in these respects.

At operation 510, patient 410 can sit, recline, or lie on sensor substrate 110. Sensor substrate 110 can obtain one or more measurements using one or more internal sensors 112 at operation 512. The measurements obtained by sensor substrate 110 can be processed or analyzed at operation 514. In some examples, the measurements can be processed by processor 210 of control module 116, or optionally by a processor of an external device such as smart device 228 or smart device 412, or by health care system 416. In one or more embodiments, monitoring system 100 or monitoring system 400 can be configured with processes or algorithms to process the received sensor data to detect, measure, determine, assess, or execute one or more of the following parameters:
when a patient is sleeping;
the patient's posture while sleeping;
patient movement/shifting/restlessness;
the patient's heart rate (HR);
changes in heart rate;
heart rate variability (HRV);
whether the patient has an arrhythmia such as tachycardia;
the patient's respiration rate (RR);
changes in respiration rate;
respiration rate variability (RRV)
paradoxical rhythms;
thoracic impedance;
correlate respiration rate with posture and sleep periods;
detect crackles;
detect dyspnea;
detect orthopnea;
detect wheezing;
detect rales;
the patient's weight;
changes in the patient's weight;
weight distribution such as the weight of the patient's lower legs;
pedal edema;
the patient's temperature at various locations of the patient's body;
ambient or environmental temperature at one or more locations near the patient's body;
trends of one or more of the above parameters over time;
a condition that requires urgent action such as the patient is unconscious, is not breathing, has very low blood pressure, and so on.

At operation 516, one or more external devices can obtain one or more additional measurements in addition to the measurements obtained by sensors 112 of sensor substrate 110. The one or more external devices can include the devices shown in FIG. 3 or FIG. 4. The one or more additional measurements can be processed or analyzed at operation 518. In some examples, the one or more additional measurements can be processed by processor 210 of control module 116, or optionally by a processor of an external device such as smart device 228 or smart device 412, or by health care system 416. In one or more embodiments, monitoring system 100 or monitoring system 400 can be configured with processes or algorithms for one or more of the following:
receiving patient input via a user interface;
receiving data from one or more other external devices such as home medical devices, smartphones, fitness trackers, smart speaker devices, pulse oximeters, and so on;
identifying trends of the patient input and the external device data;
identifying events or conditions from the patient input or an external device data that require an urgent response;
generating reports based on one or more of the received sensor data, received patient input, self-test results, or other devices;
generating reports with selected or customized formats geared for physicians, patients, patient guardians, clinicians, researchers, and so on.

At operation 520, a determination can be made whether a response is needed based on one or more measurements from one or more sensors 112 of sensor substrate 110 or based on one or more additional measurements from one or more external devices or sensors, or a combination thereof. If no response is needed, then method 500 can continue with operation 512. If a response is needed, then the patient 410 can be alerted at operation 522, and an alert can be transmitted to health care system 416 at operation 524. In some examples, sensor data and/or one or more sensor data reports can be transmitted to health care system 416 along with or in lieu of the alert. In one or more embodiments, monitoring system 100 or monitoring system 400 can be configured with processes or algorithms for communicating one or more of sensor data, patient input, generated reports, or alerts and alarms to:
the patient via a user interface, such as automated voice, audible alerts or alarms, visual indicators such as lights, vibrations, and so on;
the patient via other communication devices such as smartphones, for example directly via email, text, or automated voice, or via an application loaded on the smartphone;
the physician or health care provider via email or text, and so on;
a remote server configured to provide the data to other parties as configured in the server;

other Internet of Things (IoT) devices, for example a reader type device that a visiting nurse or physician can use to collect data from the system.

In some examples, the decision at operation 520 whether a response is needed can be made based on data or measurements obtained by the sensors 112 of sensor substrate 110, and optionally on one more additional measurements obtained by one or more additional sensors or devices external to sensor substrate 110, when the data or measurements indicate that the patient has experienced heart failure decompensation or is trending toward heart failure decompensation, for example having values or change in values that meet or exceed a threshold. For example, some research has shown that sleep incline, RR increase, RR being above 20 breaths per minute, high RRV, low HRV can indicate increase risk of heart failure hospitalization. In some embodiments, processor 210 of control module 116, or a processor of an external device such as smart device 412, external device 424, or health care system 416, can analyze the data or measurements from the various sensors of the monitoring system to determine whether a response is needed, for example whether to alert patient 410, alert health care provider, apply or titrate medicine or therapy 426, and so on. In some examples, the analysis can comprise determining a statistical likelihood that the patient 410 is experiencing heart failure decompensation. In some embodiments, the analysis can comprise determining a statistical likelihood that the patient 410 will imminently experience heart failure decompensation, or the patient 410 is trending toward heart failure decompensation. This likelihood can be in the form of a probability or other metric or score.

In accordance with one or more embodiments, the probability that a patient 410 is experiencing heart failure decompensation can be calculated as follows. An example probability of the patient 410 experiencing heart failure decompensation can be calculated from the following formula:

1/[1+exp(Q1+Q2+ . . . +QN)]

where Q1, Q2, QN (where N is an integer 1 or greater) can comprise any value of a measurement or data obtained by any of the sensors described herein for any patient parameter or condition discussed herein, including data or measurements from sensors 112 of sensor substrate 110. In some examples, N can be 1, 2, or three and so on. In other examples, N can be 4, 5, or 6, and so on. In yet other examples, N can comprise any integer value, and the scope of the disclosed subject matter is not limited in this respect. It should be noted that the above formula is merely one non-limiting example of how a probability of whether patient 410 is experience heart failure decompensation, and one or more other equations or formulas can be used other than the example formula above, and the scope of the disclosed subject matter is not limited in this respect. In some examples, the values can be normalized to be in a range or magnitude that is comparable with the ranges or magnitudes of values of from one or more other sensors. In some examples, the values of Q1-QN can comprise a change in value, which can be represented as a fraction or a percentage, of a measurement or data obtained by a sensor including any of the parameters measured or monitored by monitoring system 100 or monitoring system 400. For example, Q1-QN may be based on N of the following parameters: the patient's weight, the patient's limb weight, the patient's heart rate, the patient's HRV, the patient's absolute temperature, the patient's temperature relative to ambient room temperature, the patient's angle of sitting, reclining or sleeping, the restlessness of the patient, the patient's heart rate, the patient's thoracic impedance, the patient's blood pressure, the patient's blood oxygen saturation, respiration rate, the patient's RRV, the patient's transthoracic impedance, arrhythmia detection, crackle detection, dyspnea detections, orthopnea detection, wheezing detection, rales detection, or any change or fractional change thereof, and the scope of the disclosed subject matter is not limited in this respect. In some examples, Q1-QN can all have equal weight, and in other examples Q1-Q3 can be weighted differently. For example, values of Q1-QN directed to heart rate, respiration rate, arrhythmia detection, and/or HRV can be given higher weight in the formula, whereas other parameters in the formula can have a lower weight in the formula. In this context, a higher weight refers to having more influence in the value of the probability determined by the formula. Furthermore, in some embodiments, measured or sensed values or parameters that have a higher level of urgency, for example when the patient is unconscious, is not breathing, has very low blood pressure, and so on, can cause evaluation of the probability formula to be bypassed such that an immediate action or alert can be performed as the affirmative response to operation 520. In some examples, additional measures can be taken in such urgent circumstances. In another scenario, if patient 410 is wearing a wearable cardioverter defibrillator while using sensor substrate 110, the cardioverter defibrillator can initiate application of shock therapy to patient 410 in addition to providing appropriate alerts to the patient 410, health care system 416, or health care provider 418, or the administration of medicine or treatment as discussed below.

In some embodiments, the probability equation above or similar algorithm can be evaluated or calculated using processor 210 of control module, or a processor of smart device 412, external device 424, or health care system 416. In some examples, any of these processors can analyze the data or measurements from the various sensors along the patient's answers to the questions or queries from health care provider 418. Some of the above values can be obtained from various external devices such as using ECG electrodes 216 of an ECG device used by the patient 410 to determine heart rate, arrythmia detection, thoracic impedance, or respiration. If it is determined that the probability meets or exceeds a threshold value, then a determination can be made at operation 520 that a response is needed, an appropriate alert can be generated or transmitted at operation 522 or operation 524.

At operation 526, treatment information can be received from health care system 416. Monitoring system 100 or monitoring system 400 can apply the treatment or medication 426 according to the received treatment information. In some examples, the treatment or medication 426 can include titration of intravenous (IV) decongestive therapy, for example diuretics, inotropes, vasoactive drugs, and so on. See for example F. Roosevelt Gilliam III et al., “Feasibility of Automated Heart Failure Decompensation Detection Using Remote Patient Monitoring: Results from the Decompensation Detection Study”, The Journal of Innovations in Cardiac Rhythm Management, 3 (2012), 735-745, April 2012.

In some embodiments, method 500 of FIG. 5 can be implemented as machine or computer readable instructions stored on a non-transitory machine or computer readable medium such as memory 214 of FIG. 2. Such instructions can cause processor 210 to execute method 500 to implement any one or more of the operations of method 500 as discussed herein. In some embodiments, monitoring system 100 or monitoring system 400 can be configured to monitor and detect other conditions or ailments of patient 410. For example, sensor data related to patient movement, sounds, or posture can be used to detect seizures or tremors. Furthermore, monitoring system 100 or monitoring system 400 can include one or more sensors to detect parameters related to the patient's sleeping times, movement, gait, waking time in bed, and so on, to detect Alzheimer's disease. It should be noted that these are merely examples of other conditions or ailments that can be detected with monitoring system 100 or monitoring system 400, and the scope of the disclosed subject matter is not limited in these respects.

Although the claimed subject matter has been described with a certain degree of particularity, it should be recognized that elements thereof may be altered by persons skilled in the art without departing from the spirit and/or scope of claimed subject matter. It is believed that the subject matter pertaining to heart failure decompensation monitoring and many of its attendant utilities will be understood by the forgoing description, and it will be apparent that various changes may be made in the form, construction and/or arrangement of the components thereof without departing from the scope and/or spirit of the claimed subject matter or without sacrificing all of its material advantages, the form herein before described being merely an explanatory embodiment thereof, and/or further without providing substantial change thereto. It is the intention of the claims to encompass and/or include such changes.

1. A sensor substrate to detect heart failure decompensation, comprising:
a substrate structure, wherein the substrate structure is configured to have a patient sit, recline, or lie on a surface of the substrate structure;
one or more sensors in the substrate structure, wherein the one or more sensors are configured to detect a plurality of parameters of the patient when the patient is in contact with the substrate structure;
an interface in the substrate structure to receive an output from the one or more sensors; and
a processor, wherein the processor is configured to detect heart failure decompensation in the patient from the plurality of parameters detected by the one or more sensors when the patient is in contact with the substrate structure.

2. The sensor substrate of claim 1, further comprising a control module coupled with the interface to receive the plurality of parameters when the patient is in contact with the substrate structure, wherein the processor is in the control module.

3. The sensor substrate of claim 2, wherein the control module includes a user interface.

4. The sensor substrate of claim 2, wherein the control module includes the processor and a memory coupled to the processor.

5. The sensor substrate of claim 2, wherein the control module is configured to provide power to the sensor substrate.

6. The sensor substrate of claim 1, wherein the sensor substrate comprises a pad.

7. The sensor substrate of claim 1, wherein the sensor substrate comprises a mattress.

8-9. (canceled)

10. The sensor substrate of claim 1, further comprising a communication module to transmit the plurality of parameters from the one or more sensors to a remote device.

11. The sensor substrate of claim 1, wherein the substrate structure is portable.

12. The sensor substrate of claim 1, wherein the substrate structure is embedded in another structure.

13. The sensor substrate of claim 1, wherein:
the one or more sensors include a transmitter to transmit a signal into a thorax of the patient, and a receiver to receive an echo signal response to the transmitted signal, wherein one or more of the plurality of parameters are indicated by the received signal, and
the processor is configured to detect heart failure decompensation in the patient based at least in part on the one or more parameters indicated by the received signal.

14. The sensor substrate of claim 13, wherein the processor comprises a digital signal processor.

15. The sensor substrate of claim 13, wherein the substrate structure has a shape to facilitate positioning of the patient's body with respect to the one or more sensors in the substrate structure.

16. The sensor substrate of claim 13, wherein the substrate structure comprises a fastener to fasten the substrate structure to another structure or to the patient's body.

17. A method to detect heart failure decompensation, comprising:
obtaining one or more measurements of the patient using a sensor substrate when the patient is in contact with the sensor substrate, wherein the one or more measurements are indicative of heart failure decompensation;
determining when a response is needed by analyzing the one or more measurements, wherein a response is needed when the measurements indicate heart failure decompensation and/or a trend toward heart failure decompensation; and
transmitting an alert to a health care system when a response is needed.

18. The method of claim 17, further comprising providing an alert to the patient when a response is needed.

19. The method of claim 17, further comprising receiving a treatment instruction from the health care system.

20. The method of claim 19, further comprising providing a treatment to the patient according to the treatment instruction.

21. The method of claim 17, further comprising:
obtaining one or more additional measurements using one or more additional sensors external to the sensor substrate, wherein the one or more additional measurements indicate when the patient has heart failure decompensation; and
performing said determining by analyzing the one or more additional measurements.

22. The method of claim 17, wherein the one or more measurements are obtained when the patient is sitting on the sensor substrate.

23. The method of claim 17, wherein the one or more measurements are obtained when the patient is reclining on the sensor substrate.

24. The method of claim 17, wherein the one or more measurements are obtained with the patients is lying on the sensor substrate.

25.-36. (canceled)
